(19) United States
(12) Patent Application Publication
(10) Pub. No.: US 2023/0260594 A1
(43) Pub. Date: Aug. 17, 2023

(54) Process for preparation of neopepitope-containing vaccine agents

(71) Applicant: Evaxion Biotech A/S, Hørsholm (DK)

(72) Inventors: Thomas Trolle, Hørsholm (DK); Christian Garde, Hørsholm (DK); Michael Schantz Klausen, Hørsholm (DK); Jens Kringelum, Hørsholm (DK)

(21) Appl. No.: 18/007,061

(22) PCT Filed: Jul. 30, 2021
(86) PCT No.: PCT/EP2021/071380

§ 371 Date: Jan. 27, 2023

(30) Foreign Application Priority Data
Jul. 30, 2020 (EP) 20188703.1
Jan. 27, 2021 (EP) 21153781.6

(51) Int. Cl. G16B 20/20; G16B 20/50; G06N 20/00; G16H 50/30

(52) U.S. Cl. CPC G16B 20/20; G16B 20/50; G06N 20/00; G16H 50/30

(57) ABSTRACT

The present invention presents an improved method for identification of neoepitopes useful in active immunotherapy targeting malignant neoplasms. The method integrates identification of somatic variants of expression product with a balanced evaluation of such variants' 1) ability to bind MHC, 2) ability to induce immune responses, 3) clonal coverage in the tumour tissue, and 4) ability to evade immune responses. Also, the method is complemented by a method for purposive deselection of neoepitopes that could induced undesired immune response against normal cells. Also disclosed is a method for preparing immunogenic compositions, a method for treatment of cancer, and a computer system for identifying neoepitopes and neopeptides

### FIELD OF THE INVENTION

The present invention relates to the field of cancer immunotherapy. In particular, the present invention relates to improved means and methods for designing and producing anti-cancer vaccines which target neoepitopes, which are expression products from a patient's malignant cells.

### BACKGROUND OF THE INVENTION

Treatment of malignant neoplasms in patients has traditionally focussed on eradication/removal of the malignant tissue via surgery, radiotherapy, and/or chemotherapy using cytotoxic drugs in dosage regimens that aim at preferential killing of malignant cells over killing of non-malignant cells.

In addition to the use of cytotoxic drugs, more recent approaches have focussed on targeting of specific biologic markers in the cancer cells in order to reduce systemic adverse effects exerted by classical chemotherapy. Monoclonal antibody therapy targeting cancer associated antigens has proven quite effective in prolonging life expectance in a number of malignancies. While being successful drugs, monoclonal antibodies that target cancer associated antigens or antigen can by their nature only be developed to target expression products that are known and appear in a plurality of patients, meaning that the vast majority of cancer specific antigens cannot be addressed by this type of therapy, because a large number of cancer specific antigens only appear in tumours from one single patient, cf. below.

As early as in the late 1950'ies the theory of immunosurveillance was formulated and suggested that lymphocytes recognize and eliminate autologous cells—including cancer cells—that exhibit altered antigenic determinants, and it is today generally accepted that the immune system inhibits carcinogenesis to a high degree. Nevertheless, immunosurveillance is not 100% effective and it is a continuing task to develop cancer therapies where the immune system's ability to eradicate cancer cells is sought improved/stimulated.

One approach has been to induce immunity against cancer-associated antigens, but even though this approach has potential, it suffers the same drawback as antibody therapy that only a limited number of antigens can be addressed.

Many, if not all, tumours express mutations. These mutations potentially create new targetable antigens (neoantigens), which are potentially useful in specific T cell immunotherapy if it is possible to identify the neoantigens and their antigenic determinants (neoepitopes) within a clinically relevant time frame. Since it with current technology is possible to fully sequence the genome of cells and to analyse for existence of altered or new expression products, it is possible to design personalized vaccines based on neoantigens and their neoepitopes.

Multiple bioinformatic pipelines hence exist for predicting/identifying neoepitopes from patient derived sequencing data (cf. Hundal, J. et al. 2016; Bjerregaard, A. M. et al. 2017; Bais, P. et al. 2017; Rubinsteyn, A. et al. 2017; Schenck, R. O. et al. 2019). Each pipeline takes a different set of features into account when selecting or ranking neoepitopes, underscoring that the neoepitope selection problem is still unsolved.

### OBJECT OF THE INVENTION

It is an object of embodiments of the invention to provide improved methods and means for selection of immunogenic epitopes in cancer patients. that can be targeted by active immunotherapy.

### SUMMARY OF THE INVENTION

The present invention is based on the observation by the inventors that to identify key features for neoepitope effectiveness, one must understand the relevant underlying mechanisms within the cell.

Cells become cancerous by accumulating somatic variants in their genomes, causing them to grow uncontrollably. These variants are specific to the tumour cells, and thus attractive targets, in particular for immunotherapies. Accurate identification of somatic variants is a critical step in neoepitope identification. Incorrect somatic variant calling may result in i) selection of a peptide sequence that does not exist in the tumour cells or ii) selection of a peptide sequence that is also present in healthy cells.

Somatic variants in expressed genes are transcribed and translated into tumour-specific antigens.

These antigens are then processed by the antigen presentation pathway, creating neoepitopes presented as ligands by MHC molecules on the tumour cell surface. Presentation of the neoepitope by the MHC on the cell surface is a prerequisite for eliciting a T cell response. MHC binding and presentation is one of the most restrictive steps in antigen presentation and is thus an essential feature for neoepitope effectiveness.

While MHC presentation is required for eliciting an immune response, it is not in itself sufficient. To induce an anti-tumour effect, neoepitopes derived MHC ligands must also be immunogenic, e.g. by being recognized by cytotoxic CD8⁺ T cells.

Tumours are extremely heterogeneous and exhibit clonal variation, meaning cancer cells in different subsets of a tumour do not necessarily contain the same sets of somatic gene variants. Somatic variants that are present in all tumour cells are defined as “clonal”, while all other variants are “sub-clonal”. As a consequence of this, neoepitopes arising from sub-clonal will only be present in a subset of tumour cells. Targeting clonal neoepitopes has the clinical benefit of allowing activated T cells to eliminate the tumour completely instead of only targeting a subset of tumour cells.

Due to the immune pressure generated by the neoepitope treatment, there is a positive selection for tumour cells to “learn” to avoid expressed neoepitopes. To combat this, neoepitopes found in either essential genes or oncogenic drivers can be prioritized. This ensures that tumour cells that try to downregulate the neoepitope bearing genes are either not viable or not malignant.

So, in a first aspect the present invention relates to a method for identifying a set of distinct amino acid altering nucleotide mutations derived from a malignant neoplasm in an individual, the method comprising either

a) inputting genetic sequence information from cells of the malignant neoplasm and from normal cells of the individual into at least 2 distinct mutation calling models, which each generates as a result a set of identified nucleotide mutations and at least one first feature associated with such identified nucleotide mutations, and optionally appending at least one second feature generated from the said genetic information to each identified nucleotide mutation, wherein each at least one and second features if necessary is transformed into a value ≥0 and ≤1, and passing the values ≥0 and ≤1 for each identified nucleotide mutation to a machine learning model, such as an artificial neural network, which has been trained with verified mutated nucleotide sequences, and which for each identified nucleotide mutation calculates a probability that it is a nucleotide mutation specific to the malignant neoplasm, or

b) inputting genetic sequence information from cells of the malignant neoplasm and from normal cells of the individual into a machine learning model, such as an artificial neural network, wherein the machine learning model has been trained with verified mutated nucleotide sequences, and wherein the machine learning model for each identified mutated nucleotide calculates a probability that it is a nucleotide mutation specific to the malignant neoplasm; and

outputting from the machine learning model the set of distinct nucleotide mutations specific to the malignant neoplasm.

In a second aspect, the invention relates to a method for identifying at least one amino acid sequence, which constitutes a putative immunogenic neopeptide, the method comprising identifying the set of distinct amino acid altering nucleotide mutations according to the first aspect, and subsequently generating a putative neopeptide amino acid sequence, which is a subsequence of a proteinaceous expression product from the malignant neoplasm and which is encoded by a nucleic acid sequence, which comprises at least one of the distinct amino acid altering nucleotide mutations of the set, analysing the putative neopeptide for the presence of MHC ligands in the individual, where such MHC ligands must include in their respective amino acid sequences an amino acid residue encoded by a nucleotide triplet that includes at least one distinct amino acid altering nucleotide mutation of the set, and identifying each putative neopeptide as a putative immunogenic neopeptide, if analysing for presence of such MHC ligands results in a positive outcome.

In a third aspect, the invention relates to a method for identifying neoepitope containing peptides that are safe to administer to a patient, wherein each neoepitope is encoded by a nucleotide sequence comprising at least one amino acid altering nucleotide mutation, the method comprising testing the expression products or proteome from normal cells in the patient for the presence of reference any amino acid sequence, wherein
said amino acid sequence is present in a proteinaceous expression product from the patient and comprising the neoepitope, and
said amino acid sequence has a length of at least 7 amino acid residues (a practical upper limit is for the purpose of MHC Class I 11 amino acid residues, for Class II the upper limit is about 20 amino acids), and
said amino acid sequence includes as one of the at least 7 amino acid residues an amino acid altered by the at least on amino acid altering mutation; and
identifying a neoepitope as safe to administer if testing is negative.

In a fourth aspect, the invention relates to a method for determining the composition of immunogenic neopeptides comprising neoepitopes or the composition of nucleic acids encoding said immunogenic neopeptides, where the immunogenic neopeptides are derived from a malignant neoplasm, the method comprising assigning a probability score to each in a set of putative immunogenic neopeptides defined as the product of at least two of A, B, C, D, and E, wherein

each of A, B, C, D, and E is a probability score ≥0 and ≤1 and wherein

A is the probability that the putative immunogenic neopeptide's amino acid sequence comprises an amino acid encoded by a nucleotide sequence comprising a distinct amino acid altering nucleotide mutation specific to the malignant neoplasm as identified in the first aspect of the invention

B is the probability that the putative immunogenic neopeptide's amino acid sequence comprises an amino acid encoded by a nucleotide sequence comprising a distinct amino acid altering nucleotide mutation present in all cells of the malignant neoplasm as determined in the first aspect,

C is the probability that the putative immunogenic neopeptide comprises a ligand for MHC in the individual from which the malignant neoplasm is derived, as determined in the second aspect,

D is the probability that the neopeptide is immunogenic in the individual from which the malignant neoplasm is derived, as determined in the second aspect; and

E is the probability that the neopeptide is resilient toward immune evasion as determined in the second aspect,

and determining the composition by excluding from the composition any neopeptide or nucleic acid for which said product does not exceed a predefined threshold value, such as excluding those peptides where said product does not exceed 0.5.

In a fifth aspect, the invention relates to a method for preparing an immunogenic composition tailored for a patient suffering from a malignant neoplasm, the method comprising sequencing DNA and RNA from malignant cells and at least DNA from normal cells in the patient to identify a set of neopeptides, which comprise neoepitopes, derived from said malignant cells, and subsequently preparing the immunogenic composition by admixing a pharmaceutically acceptable carrier or diluent with

1) at least 1 fusion protein comprising neopeptides from the set but excluding neopeptides from the set, which are not safe to administer when evaluated by the method of the third aspect,

2) multiple neopeptides from the set but excluding neopeptides from the set, which are not safe to administer when evaluated by the method of the third aspect, or

3) at least one nucleic acid encoding the at least one fusion construct in 1) or the multiple neopeptides in 2).

In a sixth aspect, the present invention relates to a method for preparing an immunogenic composition tailored for a patient suffering from a malignant neoplasm, the method comprising sequencing DNA and/or RNA from malignant cells and at least DNA from normal cells in the patient to identify a set of neopeptides, which comprise neoepitopes, derived from said malignant cells, and subsequently preparing the immunogenic composition by admixing a pharmaceutically acceptable carrier or diluent with

i) at least 1 fusion protein comprising neopeptides from the set but excluding neopeptides from the set that are not part of the composition determined according to the firth aspect,

ii) multiple neopeptides from the set that are not part of the composition determined according to the fifth aspect, or

iii) at least one nucleic acid encoding the at least one fusion construct in i) or the multiple neopeptides in ii).

In a seventh aspect, the present invention relates to a method for treating a patient suffering from a malignant neoplastic disease, the method comprising administering an effective amount of an immunogenic composition prepared according the method of the sixth aspect of the invention.

In an eighth aspect, the invention relates to a computer or computer system. comprising

a) means for inputting and means for storing nucleic acid sequences,

b) means for inputting and means for storing a qualifier for each nucleic acid sequence input in a, said qualifier indicating whether the inputted nucleic acid sequence originates from malignant cells or non-malignant cells,

c) executable code adapted to generate and store amino acid sequences of expression products encoded by nucleic acid sequences input and stored by the means in a, and which have a qualifier indicating malignant cell origin,

d) executable code adapted to generate and store amino acid sequences of expression products encoded by nucleic acid sequences input and stored by the means in a, and which have a qualifier indicating non-malignant cell origin,

e) executable code adapted to identify amino acid sequences being part of or constituting a sequence generated and stored by the executable code in c, and not being part of or constituting a sequence generated and stored by the executable code in d,

f) executable code for tagging and/or storing each amino acid sequence identified by the executable code in e, including tagging and/or storing information identifying altered amino acid residues relative to the most similar amino acid sequence(s) present in the sequences generated and store by the executable code in d,

g) executable code, which exhaustively compares, for each amino acid sequence tagged or stored by the executable code in f, those amino acid sequences input and stored by the executable code in c, which
all have the same length X, where X is an integer ≥7,
each overlap with the amino acid sequence tagged and/or stored by the executable code in f, and
each include the altered amino acid residue for which information is tagged and/or stored in f,

with the amino acid sequences input and stored by the executable code in d,

h) executable code for outputting and/or storing amino acid sequences tagged or stored by the executable code in f while excluding those amino acid sequences for which the executable code in g results in a least one positive comparison.

### LEGENDS TO THE FIGURE

FIG. 1: Venn diagrams showing the overlap between somatic variants called by two state-of-the-art variant callers, Mutect2 and Strelka, as well as the somatic variant calling model of the invention.

Data source: (Shi, W. et al. 2018). Left: Case 3 biorep A. Right: Case 5 biorep A.

FIG. 2: Graph showing a conversion function between ligand score and probability.

FIG. 3: Graph showing an example of filtering of somatic variant calls.

FIG. 4: Graph showing examples of addition of weights to a feature probability transformation.

FIG. 5: Illustration of transformation of HLA ligand likelihood and expression of variant isoform.

FIG. 6: Box plots illustrating the relationship between 1) neoepitope quality assessed according to the present invention and 2) the clinical response exhibited by vaccinated malignant melanoma patients.

The five groups on the X-axis represent the combined neoepitope quality assessment based on all of probabilities A-D (somatic mutation probability, clonality probability, MHC ligand probability, and immunogenicity probability) as well as the neoepitope quality assessment based on the individual probabilities A-D.

FIG. 7: Box plot illustrating the relationship between the frequency of high quality neoepitopes as assessed by the present invention and the clinical response in vaccinated malignant melanoma patients.

### DETAILED DISCLOSURE OF THE INVENTION

### Definitions

A “cancer specific” antigen, is an antigen, which does not appear as an expression product in an individual's non-malignant somatic cells, but which appears as an expression product in cancer cells in the individual. This is in contrast to “cancer-associated” antigens, which also appear—albeit at low abundance—in normal somatic cells, but are found in higher levels in at least some tumour cells.

The term “adjuvant” has its usual meaning in the art of vaccine technology, i.e. a substance or a composition of matter which is 1) not in itself capable of mounting a specific immune response against the immunogen of the vaccine, but which is 2) nevertheless capable of enhancing the immune response against the immunogen. Or, in other words, vaccination with the adjuvant alone does not provide an immune response against the immunogen, vaccination with the immunogen may or may not give rise to an immune response against the immunogen, but the combined vaccination with immunogen and adjuvant induces an immune response against the immunogen which is stronger than that induced by the immunogen alone.

A “neoepitope” is an antigenic determinant (typically an MHC Class I or II restricted epitope), which does not exist as an expression product from normal somatic cells in an individual due to the lack of a gene encoding the neoepitope, but which exists as an expression product in mutated cells (such as cancer cells) in the same individual. As a consequence, a neoepitope is from an immunological viewpoint truly non-self in spite of its autologous origin and it can therefore be characterized as a tumour specific antigen in the individual, where it constitutes an expression product. Being non-self, a neoepitope has the potential of being able to elicit a specific adaptive immune response in the individual, where the elicited immune response is specific for antigens and cells that harbour the neoepitope. Neoepitopes are on the other hand specific for an individual as the chances that the same neoepitope will be an expression product in other individuals is minimal. Several features thus contrast a neoepitope from e.g. epitopes of tumour specific antigens: the latter will typically be found in a plurality of cancers of the same type (as they can be expression products from activated oncogenes) and/or they will be present—albeit in minor amounts—in non-malignant cells because of over-expression of the relevant gene(s) in cancer cells.

A “neopeptide” is a peptide (i.e. a polyamino acid of up to about 50 amino acid residues), which includes within its sequence a neoepitope as defined herein. A neopeptide is typically “native”, i.e. the entire amino acid sequence of the neopeptide constitutes a fragment of an expression product that can be isolated from the individual, but a neopeptide can also be “artificial”, meaning that it is constituted by the sequence of a neoepitope and 1 or 2 appended amino acid sequences of which at least one is not naturally associated with the neoepitope. In the latter case the appended amino acid sequences may simply act as carriers of the neoepitope, or may even improve the immunogenicity of the neoepitope (e.g. by facilitating processing of the neopeptide by antigen-presenting cells, improving biologic half-life of the neopeptide, or modifying solubility).

The term “amino acid sequence” is the order in which amino acid residues, connected by peptide bonds, lie in the chain in peptides and proteins. Sequences are conventionally listed in the N to C terminal direction.

“An immunogenic carrier” is a molecule or moiety to which an immunogen or a hapten can be coupled in order to enhance or enable the elicitation of an immune response against the immunogen/hapten. Immunogenic carriers are in classical cases relatively large molecules (such as tetanus toxoid, KLH, diphtheria toxoid etc.) which can be fused or conjugated to an immunogen/hapten, which is not sufficiently immunogenic in its own right—typically, the immunogenic carrier is capable of eliciting a strong T-helper lymphocyte response against the combined substance constituted by the immunogen and the immunogenic carrier, and this in turn provides for improved responses against the immunogen by B-lymphocytes and cytotoxic lymphocytes. More recently, the large carrier molecules have to a certain extent been substituted by so-called promiscuous T-helper epitopes, i.e. shorter peptides that are recognized by a large fraction of HLA haplotypes in a population, and which elicit T-helper lymphocyte responses.

A “T-helper lymphocyte response” is an immune response elicited on the basis of a peptide, which is able to bind to an MHC class II molecule (e.g. an HLA class II molecule) in an antigen-presenting cell and which stimulates T-helper lymphocytes in an animal species as a consequence of T-cell receptor recognition of the complex between the peptide and the MHC Class II molecule presenting the peptide.

An “immunogen” is a substance of matter which is capable of inducing an adaptive immune response in a host, whose immune system is confronted with the immunogen. As such, immunogens are a subset of the larger genus “antigens”, which are substances that can be recognized specifically by the immune system (e.g. when bound by antibodies or, alternatively, when fragments of the are antigens bound to MHC molecules are being recognized by T-cell receptors) but which are not necessarily capable of inducing immunity—an antigen is, however, always capable of eliciting immunity, meaning that a host that has an established memory immunity against the antigen will mount a specific immune response against the antigen.

A “hapten” is a small molecule, which can neither induce or elicit an immune response, but if conjugated to an immunogenic carrier, antibodies or TCRs that recognize the hapten can be induced upon confrontation of the immune system with the hapten carrier conjugate.

An “adaptive immune response” is an immune response in response to confrontation with an antigen or immunogen, where the immune response is specific for antigenic determinants of the antigen/immunogen—examples of adaptive immune responses are induction of antigen specific antibody production or antigen specific induction/activation of T helper lymphocytes or cytotoxic lymphocytes.

A “protective, adaptive immune response” is an antigen-specific immune response induced in a subject as a reaction to immunization (artificial or natural) with an antigen, where the immune response is capable of protecting the subject against subsequent challenges with the antigen or a pathology-related agent that includes the antigen. Typically, prophylactic vaccination aims at establishing a protective adaptive immune response against one or several pathogens.

“Stimulation of the immune system” means that a substance or composition of matter exhibits a general, non-specific immunostimulatory effect. A number of adjuvants and putative adjuvants (such as certain cytokines) share the ability to stimulate the immune system. The result of using an immunostimulating agent is an increased “alertness” of the immune system meaning that simultaneous or subsequent immunization with an immunogen induces a significantly more effective immune response compared to isolated use of the immunogen.

The term “polypeptide” is in the present context intended to mean both short peptides of from 2 to 50 amino acid residues, oligopeptides of from 50 to 100 amino acid residues, and polypeptides of more than 100 amino acid residues. Furthermore, the term is also intended to include proteins, i.e. functional biomolecules comprising at least one polypeptide; when comprising at least two polypeptides, these may form complexes, be covalently linked, or may be non-covalently linked. The polypeptide(s) in a protein can be glycosylated and/or lipidated and/or comprise prosthetic groups.

### Specific Embodiments of the Invention

### 1^{st} Aspect

The method of the first aspect of the invention provides for improved “mutation calling”, i.a. an improvement in handling the identification of genetic differences between malignant tissue/cells and normal tissue/cells. The core of this aspect is that instead of providing a binary output, which merely identifies a sequence as a neopeptide or neoepitope, a probability is outputted together with an amino acid sequence. In turn, this allows for a convenient way of prioritizing the output by simply selecting the most relevant peptides rather than being forced to consider all peptides identified as equally good vaccine candidates.

In the event option a is carried out, the first and second features are transformed into a probability value if this is not already so. In the event option b is carried out, the machine learning model may be fed with data corresponding the at least second features used in option a.

At any rate, it is preferred that the outputted distinct nucleotide mutations specific to the malignant neoplasm in the set are
prioritized relative to the calculated probabilities, and/or
paired with their respective calculated probabilities and/or
all have a calculated probability, which exceeds a threshold value, such as a threshold value of 0.5 (50%).

The latter possibility hence screens out peptides that for all practical purposes must be considered irrelevant as vaccine agents, whereas the remaining peptides (prioritized and/or paired with probabilities) are then subject to further evaluation/selection.

The at least one first and/or second feature is typically selected from the group consisting of tumour variant coverage, normal variant coverage, tumour variant allele frequency, normal variant allele frequency, tumour read mapping quality, normal read mapping quality, tumour base quality, and normal base quality, but any measurable quality that can have an influence on the fidelity of the finding that an amino acid sequence is a neoepitope specific for the cancer can in principle be part of the information evaluated

On important characteristic based on which a nucleotide mutation can be identified/selected is it “clonality status”, i.e. the extent to which the mutation is present in all cells in the malignant tumour or only in one or a few clonal lines. It goes without saying that a mutation, which only is found in a limited number of the malignant cells is incapable of raising an immune response against all cells in the tumour. Therefore, the composition of any vaccine targeting the cancer should preferably include expression products from nucleotide mutated sequences that together target the entire spectrum of malignant cells in the cancer. Therefore, the first aspect preferably entails that each nucleotide mutation in the set of distinct nucleotide mutations specific to the malignant neoplasm is evaluated for clonality status, as this allows rational composition of a vaccine that targets all malignant cells. Therefore, the clonality status is utilized to prioritize the list in order to predominantly include the distinct nucleotide mutations specific to the malignant neoplasm that are present in a large proportion of the cells of the malignant tumour; or at the very least, when taken together target a maximum number of clones in the malignant cell population.

### 2^{nd} Aspect

Analysing for presence for MHC ligands preferably comprises integrating prediction of MHC binding with an expression level score of the proteinaceous expression product, in order to avoid targeting of protein with very low expression levels. Typically, the expression level score is calculated from an RNA expression level, and in most practical embodiments the RNA expression level is the RNA expression level of the amino acid altering nucleotide mutation.

When quantifying the expression levels of expressed genes that encode neoepitopes, it is important to remember that most genes are present in at least 2 copies (alleles) in the cancer cells and that these alleles may not necessarily be expressed in equal amounts. Most neoepitopes arise from random mutations in the cancer cells' genomes. Thus, for many (or most) neoepitopes, only a subset—and often only 1—of the alleles contains the amino acid altering somatic mutation that gives rise to a neoepitope.

Standard state-of-the-art tools for quantifying RNA expression levels, such as RSEM, do not distinguish between multiple alleles of the same gene. One can nevertheless calculate the mutation-specific expression levels in multiple ways:
1) Instead of calculating expression levels on a per gene- or transcript basis, one could calculate expression levels per genomic/transcriptomic position
2) One can modify the per gene/transcript expression levels by the variant allele frequency (VAF) of the somatic mutation observed in the RNA sequencing data, normalized by the VAF observed in the DNA sequencing data:

$$\frac{V A {F}_{R N A}}{V A {F}_{D N A}} \times Expression$$

Using these approaches to quantify expression levels provides a further certainty that the expression level score is accurate, thus minimizing the risk that an irrelevant protein is targeted.

So, in an embodiment of the 2^{nd} aspect of the invention, the expression level score is calculated per genomic/transcriptomic position or wherein the expression level score is modified by adjusting for the ratio VAF_{RNA}/VAF_{DNA}, where VAF denotes frequency of the variant allele comprising the nucleic acid sequence, which comprises at least one of the distinct amino acid altering nucleotide mutations of the set discussed in the first aspect of the invention.

In addition to MHC binding, it is also of value to determine/assess the immunogenicity of the putative immunogenic neopeptide. In practice, this can be done by any one or more or of the following procedures: assessment of presence of T-cell receptor binding amino acid residues when the putative immunogenic neopeptide is part of a peptide-MHC complex; assessment of stability of the complex between MHC and the putative immunogenic neopeptide (for this purpose, use can be made of the stability determination methods disclosed in the present applicant's currently unpublished European patent applications 20185772.9 and 20180876.3), assessment of similarity between the putative immunogenic neopeptide and autologous peptides of the individual (similarity should be avoided, cf. also the third aspect herein); assessment of similarity between one the one hand complexes of MHC and putative immunogenic neopeptide and on the other hand complexes of MHC and autologous peptides of the individual; and assessment by a convolutional neural network architecture to unlock further sequential features that influence immunogenicity.

Yet one further important feature to take into consideration is the prolonged relevance as a target. A peptide immunogen, which is derived from a protein, which is “open for further mutation” may become irrelevant in a vaccine, since the intended target can escape the immune response induced. Therefore, each putative immunogenic neopeptide is preferably also evaluated for its resilience towards immune evasion. This evaluation for resilience may include a determination of whether the putative immunogenic neopeptide arises from an oncogenic driver mutation and/or is located in an expression product essential for cell survival and/or solely associate with an HLA that is lost or suppressed by the tumour—in the 2 first cases, further mutations in the same protein in the malignant cells would be detrimental to the tumour meaning that neopeptides targeting such proteins are more likely to remain relevant as an immunogen. In the latter case, the opposite is the case: even though the neopeptide identified fulfils all criteria for being an excellent immunogen, it will be ineffective in the patient because peptides from the corresponding target are not presented in an MHC context. As it turns out, it is therefore also of high relevance to HLA type the malignant cells to ensure that neopeptides comprise de facto neoepitopes in the patient

### 3^{rd} Aspect

This aspect has as its specific aim to provide neopeptides that exhibit a high degree of safety. This aspect can be combined with the method of the second aspect as it simply aims at reducing the final number of candidate vaccine peptides to avoid potentially harmful peptides.

When a neoepitope has been identified, it will in the simplest case contain 1 single amino acid change relative to a peptide excised from the same protein in a normal cell. In the case of a neoepitope of 8 amino acid residues, the peptide can be described as:

ABCXEFGHI—where all letters symbolize some amino acid and where the X is the mutated amino acid.

In the cancer cell, such a peptide will be part of a larger protein, for instance one having the partial sequence:

. . . KLMNABCXEFGHIST . . .

To ensure that no normal cells are potentially targeted by the immune response induced by ABDXEFGHI, the normal cells' expression products/transcriptome are compared with all the 8 amino acid sequences from the malignant that include the X: KLMNABCX, LMNABCXE, MNABCXEF, NABCXEFG, ABCXEFGH, BCXEFGHI, CXEFGHIS, and XEFGHIST. Only if none of these sequences are found in the normal cells, the peptide ABCXEFGH will be considered safe.

The at least 7 amino acids can be 8, 9, 10, 11, 12, 13, 14, 15, or even higher number of amino acids, depending on the length and type (Class I or II) of the epitope. However, from a safety point of view, the lower amount of amino acids will be preferred, since this will exclude the largest number of potentially harmful vaccine agents.

### 4^{th} Aspect

This aspect aims at providing a reproducible method for composing a vaccine that includes neoepitopes and takes advantage of the methods of aspects 1-3. In brief, by consistently utilising probabilities for each of the features it is of relevance to take into consideration for each candidate peptide, it is achieved that selection of the final product to be administered to the patient integrates virtually all available knowledge that can influence the suitability of each peptide.

While one preferred embodiment entails that the probability product of all of A-E is calculated, the product of at least 2 of A, B, C, D, and E can generally be selected from the group of products of A and B, A and C, A and D, A and E, B and C, B and D, B and E, C and D, C and E, D and E, A and B and C, A and B and D, A and B and E, A and C and D, A and C and E, A and D and E, B and C and D, B and C and E, B and D and E, C and D and E, A and B and C and D, A and B and C and E, A and B and D and E, A and C and D and E, B and C and D and E, and A and B and C and D and E.

To finally arrive at the composition of peptides to include in the vaccine agent, the neopeptides in the composition are preferably those that have a probability score, which is among the top 50 (i.e. in absolute number, e.g. top 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, or 10), and/or have a probability score among the top 50%, such as the top 45%, the top 40%, the top 35%, the top 30%, the top 25%, the top 20%, the top 15%, and the top 10%.

In line with the above, it is preferred that the method of the fourth aspect is combined with the method of the 3^{rd} aspect to exclude potentially harmful peptides from the final composition or agent.

### 5^{th} and 6^{th} Aspects

Both these aspects relate to the practical preparation of a vaccine composition, taking either the 3^{rd} aspect as a starting point or the method of the 4^{th} aspect. In both cases, the immunogenic composition prepared will comprise standard component for a vaccine as those that are well-known in the art. The compositions prepared according to the invention thus typically contain an immunological adjuvant, which is commonly an aluminium based adjuvant or one of the other adjuvants described in the following:

Adjuvants to enhance effectiveness of an immunogenic composition include, but are not limited to: (1) aluminum salts (alum), such as aluminum hydroxide, aluminum phosphate, aluminum sulfate, etc; (2) oil-in-water emulsion formulations (with or without other specific immunostimulating agents such as muramyl peptides (see below) or bacterial cell wall components), such as for example (a) MF59 (WO 90/14837; Chapter 10 in Vaccine design: the subunit and adjuvant approach, eds. Powell & Newman, Plenum Press 1995), containing 5% Squalene, 0.5% Tween 80, and 0.5% Span 85 (optionally containing various amounts of MTP-PE, although not required) formulated into submicron particles using a microfluidizer such as Model 110Y microfluidizer (Microfluidics, Newton, Me.), (b) SAF, containing 10% Squalane, 0.4% Tween 80, 5% pluronic-blocked polymer L121, and thr-MDP, either microfluidized into a submicron emulsion or vortexed to generate a larger particle size emulsion, and (c) Ribi adjuvant system (RAS), (Ribi Immunochem, Hamilton, Mont.) containing 2% Squalene, 0.2% Tween 80, and one or more bacterial cell wall components from the group consisting of monophosphoryl lipid A (MPL), trehalose dimycolate (TDM), and cell wall skeleton (CWS), preferably MPL+CWS (Detox™) ; (3) saponin adjuvants such as Stimulon™ (Cambridge Bioscience, Worcester, Mass.) may be used or particles generated therefrom such as ISCOMs (immunostimulating complexes); (4) Complete Freund's Adjuvant (CFA) and Incomplete Freund's Adjuvant (IFA); (5) cytokines, such as interleukins (eg. IL-1, IL-2, IL-4, IL-5, IL-6, IL-7, IL-12, etc.), interferons (eg. gamma interferon), macrophage colony stimulating factor (M-CSF), tumor necrosis factor (TNF), etc.; and (6) other substances that act as immunostimulating agents to enhance the effectiveness of the composition.

As mentioned above, muramyl peptides include, but are not limited to, N-acetyl-muramyl-L-threonyl-D-isoglutamine (thr-MDP), N-acetyl-normuramyl-L-alanyl-D-isoglutamine (nor-MDP), N-acetylmuramyl-L-alanyl-D-isoglutaminyl- L-alanine-2″-2′-dipalmitoyl-sn-glycero-3-hydroxyphosphoryloxy)-ethylamine (MTP-PE), etc.

The immunogenic compositions (eg. the immunising antigen or immunogen or polypeptide or protein or nucleic acid, pharmaceutically acceptable carrier (and/or diluent and/or vehicle), and adjuvant) typically will contain diluents, such as water, saline, glycerol, ethanol, etc. Additionally, auxiliary substances, such as wetting or emulsifying agents, pH buffering substances, and the like, may be present in such vehicles.

Pharmaceutical compositions can thus contain a pharmaceutically acceptable carrier. The term “pharmaceutically acceptable carrier” refers to a carrier for administration of a therapeutic agent, such as antibodies or a polypeptide, genes, and other therapeutic agents. The term refers to any pharmaceutical carrier that does not itself induce the production of antibodies harmful to the individual receiving the composition, and which may be administered without undue toxicity. Suitable carriers may be large, slowly metabolized macromolecules such as proteins, polysaccharides, polylactic acids, polyglycolic acids, polymeric amino acids, amino acid copolymers, and inactive virus particles. Such carriers are well known to those of ordinary skill in the art.

Pharmaceutically acceptable salts can be used therein, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients is available in Remington's Pharmaceutical Sciences (Mack Pub. Co., N.J. 1991).

Typically, the immunogenic compositions are prepared as injectables, either as liquid solutions or suspensions; solid forms suitable for solution in, or suspension in, liquid vehicles prior to injection may also be prepared. The preparation also may be emulsified or encapsulated in liposomes for enhanced adjuvant effect, as discussed above under pharmaceutically acceptable carriers.

Immunogenic compositions used as vaccines comprise an immunologically effective amount of the relevant immunogen, as well as any other of the above-mentioned components, as needed. By “immunollogically effective amount”, it is meant that the administration of that amount to an individual, either in a single dose or as part of a series, is effective for treatment or prevention. This amount varies depending upon the health and physical condition of the individual to be treated, the taxonomic group of individual to be treated (eg. nonhuman primate, primate, etc.), the capacity of the individual's immune system to synthesize antibodies or generally mount an immune response, the degree of protection desired, the formulation of the vaccine, the treating doctor's assessment of the medical situation, and other relevant factors. It is expected that the amount of immunogen will fall in a relatively broad range that can be determined through routine trials. However, for the purposes of protein vaccination, the amount administered per immunization is typically in the range between 0.5 μg and 500 mg (however, often not higher than 5,000 μg), and very often in the range between 10 and 200 μg.

The immunogenic compositions are conventionally administered parenterally, eg, by injection, either subcutaneously, intramuscularly, or transdermally/transcutaneously (cf. eg. WO 98/20734). Additional formulations suitable for other modes of administration include oral, pulmonary and nasal formulations, suppositories, and transdermal applications. In the case of nucleic acid vaccination and antibody treatment, also the intravenous or intraarterial routes may be applicable.

Dosage treatment may be a single dose schedule or a multiple dose schedule, for instance in a prime-boost dosage regimen or in a burst regimen. The vaccine may be administered in conjunction with other immunoregulatory agents as may be convenient or desired.

### 7^{th} Aspect

Also this aspect follows standard procedures well known in the art; when the precise composition and format of a vaccine has been determined as set forth above, the invention relies generally on methods well known to the medical practitioner for inducing immunity and follow up on patients. This also entails dosing of the vaccines (which in the case protein/peptide based vaccines typically entails administration of between 0.5 μg and 500 μg per dosage, typically provided as at least a priming dosage followed by one or several booster immunizations.

### 8^{th} Aspect

This aspect relates to a computer or computer system, which implements the methods described in aspects 1-4.

The means for inputting nucleic acid sequences and/or qualifiersis/are typically selected from any device for inputting data into a computer memory or storage medium: in principle, a simple keyboard connected to the computer can serve this purpose, but typically nucleic acid sequence data will be read from an external data carrier or data source by a connected disk drive or other data carrier (a memory stick, memory card, network associated storage) or via a network or internet connection and a suitable protocol for file transfer (FTP, FTPS, SFTP, CSP, HTTP or HTTPS, AS2, 3-, and -4, or PeSIT). Likewise, the means for storing nucleic acid sequences can be any convenient data carrier or storage medium (a hard drive, a solid state hard drive, a memory stick) but also directly in the memory (RAM) of the computer or computer system. The storage format can be any convenient format such as in the form of records in a relation database (both row-oriented and column-oriented), an object database, but also as entries in text files (e.g. as comma separated values or a suitable XML format, or as a simple file system or other similar root-and-tree structure.

The executable code(s) in the computer or computer system is capable of accessing the linked input devices and storage media as well as the computer working memory in ode rot perform the necessary operations of encoding amino acid sequences, sorting and comparing amino acid sequences etc.

### Further Disclosure Relating to the Invention

### Identification of Somatic Variants

Germline mutations, contamination of normal cells in tumour samples and noise from sequencing machines makes identification of somatic mutations difficult. Nonetheless, identification of somatic mutations or somatic variants is a widely studied problem and several tools for calling somatic variants have been developed. Mutect (Cibulskis, K. et al. 2013), Mutect2 (Cibulskis, K. et al. 2013), Strelka (Kim, S. et al. 2018), Varscan2 (Koboldt, D. C. et al. 2012), SomaticSniper (Larson, D. E. et al. 2012), LoFreq* (Wilm, A. et al. 2012), SNVSNiffer (Liu, Y. et al. 2016) and Shimmer (Hansen, N. F. et al. 2013) constitute but a few (Xu et al. 2018). However, due to the complexity of the problem no perfect solution has yet been found.

The present invention presents a somatic variant calling model, which utilizes a machine learning model to assign a probability of “true somatic variant” to a list of potential somatic variants. The list is generated as the collection of variants called by one of four existing variant callers, Mutect2, Strelka, SNVSNiffer and LoFreq*. However, the present invention is not limited to the use of these variant callers, as the somatic variant calling model can be used in combination with any set of variant callers, including a model trained only on genomic features without features from a variant caller.

For each potential somatic variant, features from the genomic alignment are extracted alongside features from the somatic variant callers. These genomic alignment features include, but are not limited to, tumour variant coverage (i.e. number of reads supporting the variant), normal variant coverage, tumour variant allele frequency (VAF), normal VAF, tumour read mapping quality, normal read mapping quality, tumour base quality and normal base quality etc. Other features can be appended to the set to potentially improve performance.

Features specific to variant callers are also extracted where available. Different variant callers will produce different features with information but will usually include a fail/pass descriptor as well as a calculated probability or score value for each somatic variant.

Each feature is transformed into a value ≥0 and ≤1. The method of the invention uses a linear transformation with a predefined minimum and maximum value, however other functions can be used to transform input features. Choice of transformation will be dependent on choice of machine learning model, in order to maximize performance.

$$capped\_exp ( x ) = \{ \begin{matrix}{10}^{\frac{x - c}{d}} , x < c \\ 1 , x \geq c\end{matrix} sigmoid ( x ) = \frac{1}{1 + {e}^{- a \cdot ( x - d )}} binary ( x ) = \{ \begin{matrix}0 , x < c \\ 1 , x \geq c\end{matrix}$$

The set of transformed features is passed to a machine learning model which calls somatic variants. This could for example be an artificial neural network or a gradient boosting machine that output values between 0 and 1. A separate training set is used to calibrate the model to transform the output into a probability representing the probability of a given mutation being a true somatic variant (see Example 1).

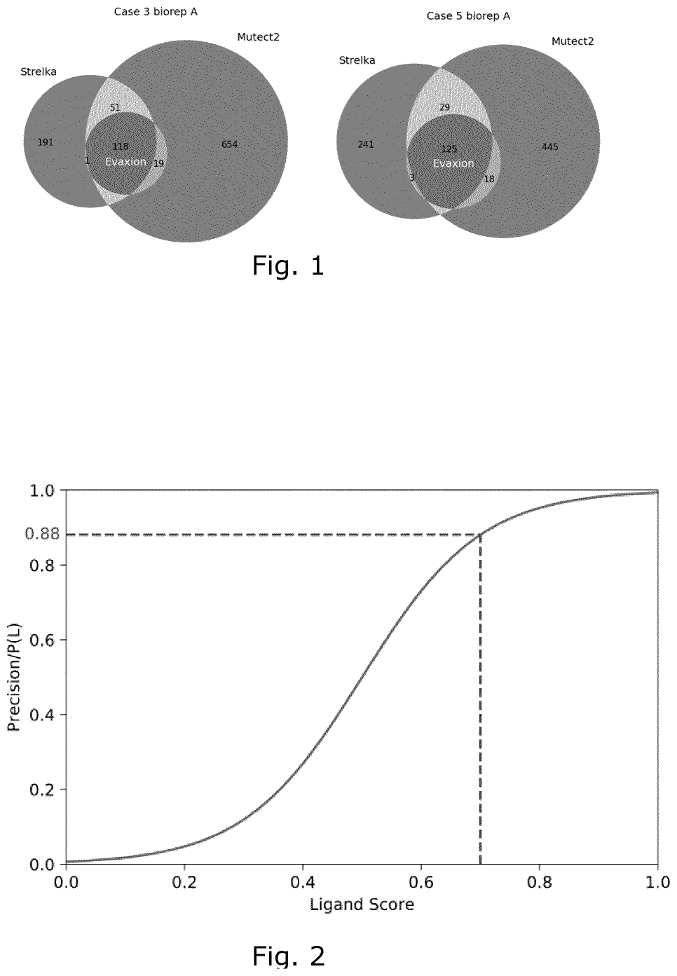

The impact of applying the current somatic variant calling model can be seen in FIG. 1. The model has been tuned to be more precise and specific with regards to which variants it calls. FIG. 1 shows two example where whole exome sequencing obtained data from Shi, W. et al. 2018 (Cases 3 and 5) have been subjected to the mutation calling routines in Strelka and Mutect2. The “case 3” data provided 361 somatic variants identified by Strelka, 842 identified by Mutect 2, with an overlap of 169 somatic variants. Application of the method of the present invention reduced this set of 169 common somatic variants to 118 regarded as true somatic variants characteristic for the tumour, plus 1 true somatic variant called only by Strelka and 19 called only by Mutect 2, a total of 138 somatic variants identified.

Likewise the “case 5” data provided 398 somatic variants identified by Strelka, 617 identified by Mutect 2, with an overlap of 154 somatic variants identified by both. Application of the method of the present invention reduced this set of 154 commonly identified variants to 125. 18 further somatic variant identified by Mutect2 and 3 identified by Strelka was also identified by the method of the invention, thus providing a total of 146 true somatic variants characteristic for the tumour.

As such the somatic variants called by the model are often a subset of high confidence somatic variants that have been identified by several of the other variant callers.

### Identification of Ligands Presented by MHC

First, somatic- and germline variants are annotated using a variant annotation tool such as VEP (McLaren, W. et al. 2016) or SnpEff (Cingolani, P. et al. 2012) and filtered to the subset annotated to impose an amino acid change i.e. the non-synonymous variants. The annotated amino acid changes are introduced into the corresponding reference protein sequence resulting in a tumour-specific protein sequence. Finally, a neopeptide sequence of 27 amino acids is extracted around each amino acid change caused by a somatic variant (13 amino acids on each side). These neopeptide sequences are subjected to prediction for hosting MHC ligands presented by the patient's HLAs.

This is done by generating appropriately sized amino acid oligomers from the neopeptide sequence and, for each oligomer, predicting the likelihood of antigen presentation against the respective HLAs. An appropriate oligomer size is defined by the relevant HLA molecule length preference, which could be 8, 9, 10 and 11 for HLA molecules belonging to MHC class I and 13 through 19 for HLA molecules belonging to MHC class II.

For the set of HLA molecules belonging to each MHC class, the best prediction represents the likelihood of the neopeptide being presented by the tumour cells or professional antigen presenting cells. This prediction is integrated with the RNA-seq derived expression level of the variant isoform of the neopeptide's source protein and transformed to a probability of antigen presentation. Neural network models for predicting the HLA ligand likelihood of a given peptide is developed based on peptide-MHC interaction data e.g. binding affinity data and MHC ligands discovered through immunopeptidomics. The neural network models take as input
BLOSUM encoded processing motifs defined as the first 3 amino acids and 3 last amino acids in the peptide.
BLOSUM encoded 9-mer peptide binding motifs
   For peptides shorter than 9 amino acids, binding motifs are generated by inserting stretches of wildcard amino acids after each amino acid in the peptide.
   For peptides of length 9 amino acids, the binding motif is given by the peptide sequence.
   For peptides longer than 9 amino acids, binding motifs are defined as 9-mers hosted by the peptide or 9-mer motifs are defined by introducing deletions in the peptide sequence (i.e. removing stretches of amino acids to reduce the peptide sequence to 9 amino acids).
The peptide length denoted L, which is transformed as
1/(1+e^{0.5×(L-9)}) for HLA molecules belonging to MHC class I
1/(1+e^{0.5×(L-15)}) for HLA molecules belonging to MHC class II
The start and end position of the 9-mer binding motif in the peptide.

One could envision applying other machine learning frameworks, e.g. combinations of convolutional neural networks, autoencoders, recursive neural networks and probabilistic learning models.

### Selection of Immunogenic Peptides

Several factors can be described that explains the difference between immunogenic and non-immunogenic peptide-MHC complexes (“pMHC”) (Calis, J. J. A. et al. 2013). First and foremost, the pMHC should be recognized by T cell receptors. Second, the pMHC should be abundant, with higher binding affinity, binding stability and expression levels of both MHC and precursor protein increasing the frequency of event of recognition. Third, the immune response following recognition of a pMHC by a T cell may be blocked/inhibited by regulatory processes.

Models (e.g. neural networks) can be trained that predicts whether a peptide-ligand alone or a pMHC is immunogenic. The following methods can be envisioned that incorporates the factors above into the model:
Position specific peptide sequence encoding: The peptide sequence can be encoded or embedded in a way that discrete categorical amino acids are represented as position specific numerical vectors. The peptide sequence encoding guides the modelling of T cell receptor binding to certain amino acids in the peptide chain (e.g. the importance of certain central amino acids protruding from the MHC complex).
Position specific MHC sequence encoding: The whole, or parts of, the MHC sequence can be encoded or embedded in a way so that discrete categorical amino acids are represented as position specific numerical vectors. The MHC sequence encoding guides the modelling of the T cell receptor binding to certain amino acids in the MHC chains (e.g. the importance to amino acids of particular chains in close proximity to the T cell receptor) and guides the interdependency of MHC amino acids and peptide amino acid with T cell binding.
The measured or predicted stability of the pMHC can be inputted to the model and help guide the modelling of immunogenicity with the abundance of the pMHC factored in.
A metric that describes the similarity to self-peptide or self-pMHC can be inputted to the model to help guide the modelling of regulatory processes impacting immunogenicity. Such a metric can be numerical values or encoded categorical variables that describe the similarity to relevant targets, such as wildtypes of neoantigens, immunopeptidomes of human precursor proteins or entire human proteomes.
A convolutional neural network architecture can unlock important abstract sequential features underneath the positional amino acids of either the peptide or MHC sequence that guides the modelling of immunogenicity.

Immunogenicity can be measured in assays such as tetramer/multimer stainings, ELISPOT, ICS etc.

The transformation of prediction scores to the immunogenicity probabilities is defined by benchmarking the predictor on an appropriate immunogenicity dataset that reflects the use case, i.e. data with readouts from the above defined assays. Predictions are made on the benchmarking dataset. After sorting the predictions, the precision is computed in moving windows along the prediction scores. A smooth and monotonically increasing function is fitted and can be applied to calibrate the prediction scores to immunogenicity probabilities.

### Selection of Clonal Somatic Variants

A DNA or RNA sequencing readout from small-read sequencing is constituted by a series of sequencing reads, each randomly sampled from chromosomes present in the tumour biopsy, which naturally consists of cells with multiple different genotypes, including healthy cells present in the tumour tissue. An additional complication is the fact that tumour cells often rearrange their chromosomes by copying or removing large sections of chromosome. Sequencing data from a biopsy contains two outputs relevant for calculating the clonal status of a putative neoantigen: i) Sequencing depth (coverage) at each position and ii) variant allele frequency (VAF) for each variant (mutation) present in the data. From this data, the tumour purity (fraction of cells that are tumour cells in a given biopsy) and the clonality status (clonal or sub-clonal) of each putative neoantigen must be calculated.

For tumour types that consist of mainly clonal variants and are of high purity, the somatic VAFs calculated from a sample will tend to distribute around ½ times the tumour purity, as two copies of each chromosome exist on average. Thus, a simple method for detecting tumour purity is to either take the mean/median of all VAFs, or the peak of the distribution of all VAFs and multiply by 2. Clonality probability is then calculated by scaling VAFs by the purity estimate.

More complicated cases where the tumour has either a high number of chromosomal rearrangements or a high number of subclonal mutations are solved by fitting a function to the VAF and the depth data, where the output is tumour purity and copy number of genomic segments. Several algorithms make use of different functions and various ways of fitting, such as FACETS (Shen, R., and V. E. Seshan 2016), TPES (Locallo, A., D. et al. 2019), hsegHMM (Choo-Wosoba, H. et al. 2018;), Sequenza (Favero, F. et al. 2015), ASCAT (Van Loo, P. et al. 2010), ichorCNA (Adalsteinsson, V. A. et al. 2017), TITAN (Ha, G. et al. 2014), PureCN (Riester, M. et al. 2016), PhyloWGS (Deshwar, A. G. et al. 2015), PyClone (Roth, A. et al. 2014), as well as several others.

### Selection of Evasion-Resistant Neoepitopes

Neoepitopes that are resistant to immune evasion by tumour cells can be broadly divided into 3 categories a) neoepitopes arising from oncogenic driver mutations, b) neoepitopes located in genes essential for cell survival, and c) neoepitopes that solely associate with an HLA that is lost or suppressed by the tumour.

Oncogenic driver mutations are good targets because they play a critical role in driving the cells' malignancy. If a tumour cell were to lose an oncogenic driver, it would likely be less malignant. Oncogenic driver mutations can be identified from various databases such as COSMIC (Tate, J. G. et al. 2019). Here, the frequency with which certain DNA mutations or amino acid changes occur can be used as a surrogate for oncogenicity, as mutations that are significantly selected for, likely play an important role.

Neoepitopes in essential genes can also be prioritized, as a tumour cell cannot downregulate the expression of these genes without this being lethal to the cell. Essential genes can be identified in a variety of experiments. One approach is to use large scale CRISPR-Cas9 loss-of-function screens (Wang, T. et al. 2015; Meyers, R. M. et al. 2017), where each gene is systematically knocked out to assess the effect on cell proliferation and survival. These types of screens have been used on large catalogues of different cancer cell lines. A gene's overall essentiality could thus simply be calculated based on the frequency with which it is essential in the tested cancer cell lines.

Loss of HLA genes or suppression of HLA expression is a described mechanism of tumour evasion (see e.g. https://pubmed.ncbi.nlm.nih.gov/29107330/). If a given HLA is lost or simply suppressed by the tumor cells, then the given HLA should be omitted from the set of HLAs that go into the MHC ligand identification step. Identification of HLA loss is done by investigating the paired tumor/normal exome-seq for depletion of reads associated with a given HLA. Suppression of HLA expression can potentially be quantified from the RNA sequencing of the tumor cells.

### EXAMPLE 1

### Probability Based Mode for Ranking of Neoepitopes

The prediction values generated for each of the above-discussed features do not necessarily follow similar distributions, making it non-trivial to combine them in a final model. One solution could be to train a model using machine learning, but the number of data points available is unfortunately extremely small and not generated in a consistent manner.

Here, we propose a model based on probabilities, where each feature prediction is converted into a probability of the prediction being true. These probabilities can then be multiplied together to give a final probability of a given neoepitope having an anti-tumour effect in the given patient.

P(neo)=P(S)*P(L)*P(I)*P(C)*P(E)

In the ideal scenario, a continuous function is created, converting a feature score into a probability of effect. An example is shown in FIG. 2. This is possible for features where high-quality evaluation datasets can be generated where an accurate precision can be calculated at various prediction thresholds.

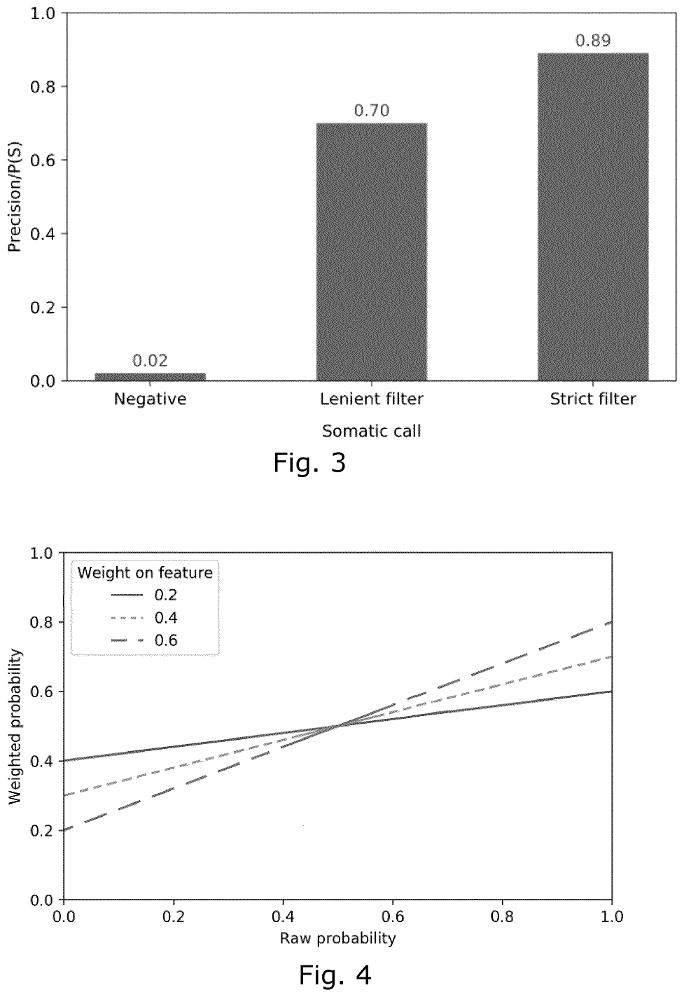

For some features, it may make sense to create a classifier rather than a tool that outputs continuous scores. One example could be in somatic variant calling, where one may want to add different filtering of various features. In this case, the precision within each category is simply calculated and used directly. An example is shown in FIG. 3. It is thus a simpler case of the example above.

For some features, it may be desirable to limit the impact it has on the final probability score. This is relevant if the evaluation dataset used is of low-quality, or if there is a high level of uncertainty associated with the returned score. In this case, it may be desirable to ensure that the weight on the feature is reduced. One could for exampe do this using the following equation:

f(x)=W*x+(1−W)

Where W is a floating-point value ≥0 and ≤1. Examples of how various weights affect the final “probability” can be seen in FIG. 4.

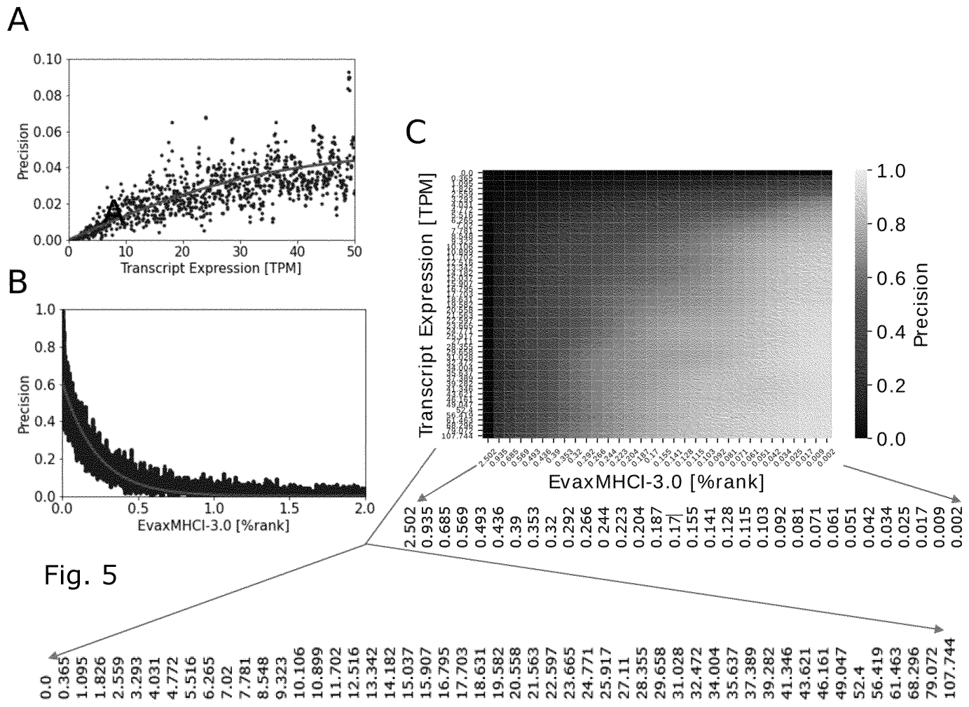

The transformation of HLA ligand likelihood and the expression of the variant isoform is developed based on RNA sequence and immunopeptidomics datasets derived from the same samples or cells grown in matched conditions. The transformation is defined by computing the precision of identifying an HLA ligand vs a random set of peptides in a 2D bin in the grid spanned by the neural network predictions along one axis and the variant isoform expression along another axis. An example is provided in FIG. 5.

More concretely this is done as follows. Initially, the neural network predictions and host transcript expression values are derived from the matched datasets. Neural network predictions and expression values are then placed on a common scale by benchmarking them individually. This is done by computing the precision in sliding windows followed by fitting of a smooth and monotonically increasing functions constitute the two univariate transformation schemes (see A and B). Next, these univariate transformations are applied to the neural network predictions and the expression values to bring them on the common scale. Afterwards, 2D bins are defined in the grid spanned by the transformed neural network predictions and the expression values. The precision is computed for each bin (see C). and the final joint probability transformation can be defined by fitting a smooth function to the computed precision landscape, here we apply a linear spline interpolation.

### EXAMPLE 2

### Evaluation of Clinical Responses Relative to the Method of the Invention

The relevance of the four features denoted A-D above under the 4^{th} aspect were tested retrospectively on a cohort of nine melanoma patients receiving an experimental peptide-based neoepitope treatment in combination with the CAF09b adjuvant, which is a liposomal adjuvant composed of N,N-dimethyl-N,N-dioctadecylammonium (Bromide salt) [DDA], monomycoloyl glycerol analogue 1 [MMG] and polyinosinic:polycytidylic acid [poly(I:C)] manufactured by Statens Serum Institut; Schmidt S. T. et al. 2020. Cells from the patients enrolled in the trial were DNA- and RNA-sequenced and neoepitopes were identified using an in silico method. Between 5-10 neoepitopes were successfully synthesized for each patient and included in the neoepitope therapy. Patients were dosed with the neoepitope therapy 6 times in total, with two weeks between each dose. The first three doses were delivered intraperitoneally and the last three intramuscularly. Imaging (PET-CT or CT scan) of each patient's tumours was performed at baseline, after three, and after six vaccinations followed by imaging every 12 weeks to assess clinical efficacy of the vaccinations. Tumours were assessed according to the RECIST v1.1 criteria. Of the nine patients, two achieved complete responses, four partial responses, one stable disease, and two had progressive disease. The objective response rate was thus 67% with six responders and three non-responders.

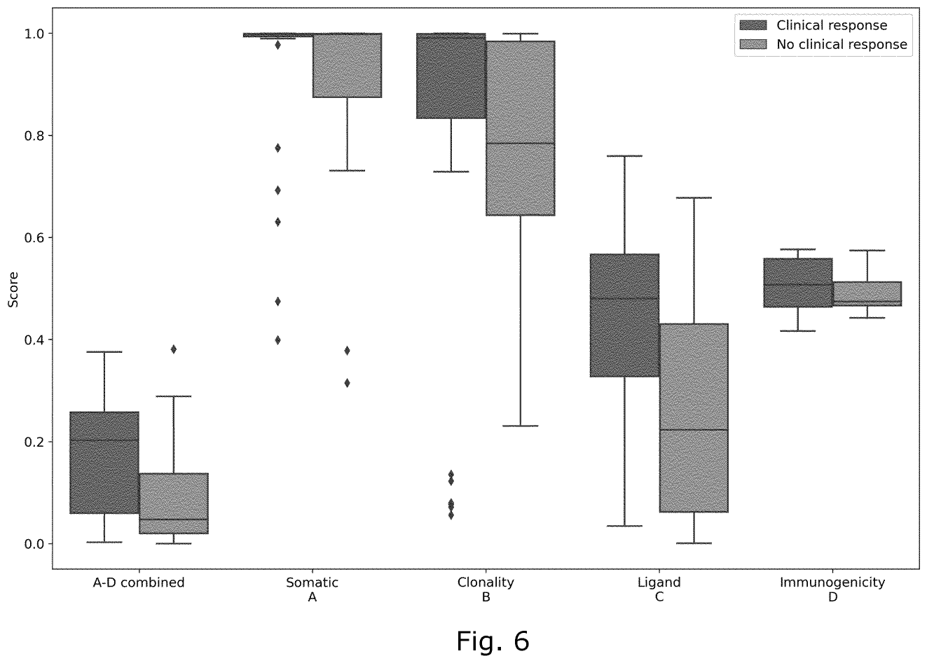

The probability scores A-D (see under the 4^{th} aspect of the invention) of the neoepitopes delivered to the responders and non-responders were compared, see FIG. 6. As apparent from this figure, each of probability scores A-D individually was able to separate the neoepitopes delivered to responders from those delivered to the non-responders, with some probabilities (B and C, i.e. the probabilities that the neoepitope is present in all tumour cells and that the neoepitope is an MHC ligand, respectively) performing better than others. The combined probability calculuation of A-D was also able to separate responder neoepitopes from non-responder neoepitopes.

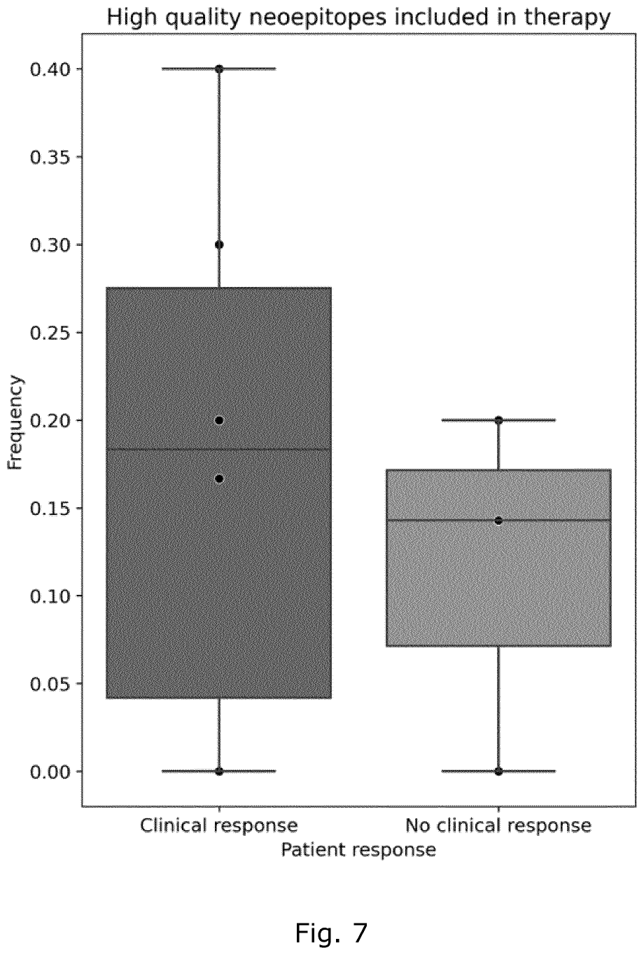

The frequency of high quality neoepitopes (high quality neoepitopes divided by total administered neoepitopes) delivered to each patient was also compared. In this case, a high quality neoepitope is defined as having a score larger than or equal to 0.5 in each of the probability scores A-D. Also here there was a distinction between the responders and non-responders, with responders having received a higher fraction of high quality neoepitopes in their therapy compared on the non-responders. See FIG. 7.

### LIST OF REFERENCES

1. Hundal, J. et al. 2016; Genome Med. 8: 11.

2. Bjerregaard, A. M. et al. 2017; Cancer Immunol. Immunother. 66: 1123-1130.

3. Bais, P. et al. 2017; Bioinformatics 33: 3110-3112.

4. Rubinsteyn, A., J. et al. 2017; Front. Immunol. 8: 1807.

5. Schenck, R. O. et al. 2019; BMC Bioinformatics 20: 264.

6. Cibulskis, K., M. et al. 2013; Nat. Biotechnol. 31: 213-219.

7. Kim, S. et al. 2018; Nat. Methods 15: 591-594.

8. Koboldt, D. C. et al. 2012; Genome Res. 22: 568-576.

9. Larson, D. E. et al. 2012; Bioinformatics 28: 311-317.

10. Hansen, N. F. et al. 2013; Bioinformatics 29: 1498-1503.

11. Xu, C. 2018; Comput. Struct. Biotechnol. J. 16: 15-24.

12. Shi, W. et al. 2018; Cell Rep. 25: 1446-1457.

13. McLaren, W. et al. 2016; Genome Biol. 17: 122.

14. Cingolani, P. et al. 2012; Fly (Austin). 6: 80-92.

15. Calis, J. J. A. et al. 2013; PLoS Comput. Biol. 9.

16. Shen, R., and V. E. Seshan 2016; Nucleic Acids Res. 44: e131.

17. Locallo, A., D. et al. 2019; Bioinformatics 35(21): 4433-4435.

18. Choo-Wosoba, H. et al. 2018; BMC Bioinformatics 19: 424.

19. Favero, F. et al. 2015; Ann. Oncol. Off. J. Eur. Soc. Med. Oncol. 26: 64-70.

20. Van Loo, P. et al. 2010; Proc. Natl. Acad. Sci. U. S. A. 107: 16910-5.

21. Adalsteinsson, V. A. et al. 2017; Nat. Commun. 8: 1324.

22. Ha, G. et al. 2014; Genome Res. 24: 1881-93.

23. Riester, M. et al. 2016; Source Code Biol. Med. 11: 13.

24. Deshwar, A. G. et al. 2015; Genome Biol. 16: 35.

25. Roth, A. et al. 2014; Nat. Methods 11: 396-8.

26. Tate, J. G. et al. 2019; Nucleic Acids Res. 47: D941-D947.

27. Wang, T. et al. 2015; Science 350: 1096-101.

28. Meyers, R. M. et al. 2017; Nat. Genet. 49: 1779-1784.

29. Wilm, A., et al. 2012; Nucleic Acids Res. 40(22): 11189-11201.

30. Liu, Y. et al. 2016; BMC Systems Biology 10: 47.

31. Schmidt S. T. et al. 2020; Pharmaceutics 2020 12(12): 1237

1. A method for identifying a set of distinct amino acid altering nucleotide mutations derived from a malignant neoplasm in an individual, the method comprising either
a) inputting genetic sequence information from cells of the malignant neoplasm and from normal cells of the individual into at least 2 distinct mutation calling models, which each generates as a result a set of identified nucleotide mutations and at least one first feature associated with such identified nucleotide mutations, and optionally appending at least one second feature generated from the said genetic information to each identified nucleotide mutation, wherein each at least one and second features if necessary is transformed into a value ≥0 and ≤1, and passing the values ≥0 and ≤1 for each identified nucleotide mutation to a machine learning model, which has been trained with verified mutated nucleotide sequences, and which for each identified nucleotide mutation calculates a probability that it is a nucleotide mutation specific to the malignant neoplasm, or
b) inputting genetic sequence information from cells of the malignant neoplasm and from normal cells of the individual into a machine learning model, wherein the machine learning model has been trained with verified mutated nucleotide sequences, and wherein the machine learning model for each identified mutated nucleotide calculates a probability that it is a nucleotide mutation specific to the malignant neoplasm; and
outputting from the machine learning model the set of distinct nucleotide mutations specific to the malignant neoplasm.

2. The method according to claim 1, wherein the outputted distinct nucleotide mutations specific to the malignant neoplasm in the set are
prioritized relative to the calculated probabilities, and/or
paired with their respective calculated probabilities and/or
all have a calculated probability, which exceeds a threshold value.

3. The method according to claim 1, wherein the at least one first and/or second feature is selected from the group consisting of tumour variant coverage, normal variant coverage, tumour variant allele frequency, normal variant allele frequency, tumour read mapping qualities, normal read mapping qualities, tumour base qualities, and normal base qualities.

4. The method according to claim 1, wherein each nucleotide mutation in the set of distinct nucleotide mutations specific to the malignant neoplasm is evaluated for clonality status.

5. The method according to claim 4, wherein the clonal probability is utilized to prioritize the list in order to predominantly include the distinct nucleotide mutations specific to the malignant neoplasm that are present in a large proportion of the cells of the malignant tumour.

6. A method for identifying at least one amino acid sequence, which constitutes a putative immunogenic neopeptide, the method comprising identifying the set of distinct amino acid altering nucleotide mutations according to claim 1, and subsequently generating a putative neopeptide amino acid sequence, which is a subsequence of a proteinaceous expression product from the malignant neoplasm and which is encoded by a nucleic acid sequence, which comprises at least one of the distinct amino acid altering nucleotide mutations of the set, analysing the putative neopeptide for the presence of MHC ligands in the individual, where such MHC ligands must include in their respective amino acid sequences an amino acid residue encoded by a nucleotide triplet that includes at least one distinct amino acid altering nucleotide mutation of the set, and identifying each putative neopeptide as a putative immunogenic neopeptide, if analysing for presence of such MHC ligands results in a positive outcome.

7. The method according to claim 6, wherein analysing for presence for MHC ligands comprises integrating prediction of MHC binding with an expression level score of the proteinaceous expression product.

8. The method according to claim 7, wherein the expression level score is calculated from an RNA expression level.

9. The method according to claim 8, wherein the RNA expression level is the RNA expression level of the amino acid altering nucleotide mutation.

10. The method according to claim 7, wherein the expression level score is calculated per genomic/transcriptomic position or wherein the expression level score is modified by adjusting for the ratio VAF_{RNA}/VAF_{DNA}, where VAF denotes frequency of the variant allele comprising the nucleic acid sequence, which comprises at least one of the distinct amino acid altering nucleotide mutations of the set.

11. The method according to claim 6, which further comprises determining the immunogenicity of the putative immunogenic neopeptide.

12. The method according to claims 11, wherein determination of immunogenicity includes one or more of
assessment of presence of T-cell receptor binding amino acid residues when the putative immunogenic neopeptide is pan of a peptide-MHC complex;
assessment of stability of the complex between MHC and the putative immunogenic neopeptide;
assessment of similarity between the putative immunogenic neopeptide and autologous peptides of the individual;
assessment of similarity between one the one hand complexes of MHC and putative immunogenic neopeptide and on the other hand complexes of MHC and autologous peptides of the individual; and
assessment by a convolutional neural network architecture to unlock further sequential features that influence immunogenicity.

13. The method according to claim 6, wherein each putative immunogenic neopeptide is further evaluated for its resilience towards immune evasion.

14. The method according to claim 13, wherein the evaluation for resilience includes a determination of whether the putative immunogenic neopeptide arises from an oncogenic driver mutation and/or is located in an expression product essential for cell survival and/or solely associate with an HLA that is lost or suppressed by the tumour.

15. A method for identifying neoepitope containing peptides that are safe to administer to a patient, wherein each neoepitope is encoded by a nucleotide sequence comprising at least one amino acid altering nucleotide mutation, the method comprising testing the expression products or proteome from normal cells in the patient for the presence of reference any amino acid sequence, wherein
said amino acid sequence is present in a proteinaceous expression product from the patient and comprising the neoepitope, and
said amino acid sequence has a length of at least 7 amino acid residues, and
said amino acid sequence includes as one of the at least 7 amino acid residues an amino acid altered by the at least on amino acid altering mutation; and
identifying a neoepitope as safe to administer if testing is negative.

16. A method for determining the composition of immunogenic neopeptides comprising neoepitopes or the composition of nucleic acids encoding said immunogenic neopeptides, where the immunogenic neopeptides are derived from a malignant neoplasm, the method comprising assigning a probability score to each in a set of putative immunogenic neopeptides defined as the product of at least two of A, B, C, D, and E, wherein each of A, B, C, D, and E is a probability score ≥0 and ≤1 and wherein
A is the probability that the putative immunogenic neopeptide's amino acid sequence comprises an amino acid encoded by a nucleotide sequence comprising a distinct amino acid altering nucleotide mutation specific to the malignant neoplasm as identified in claim 1,
B is the probability that the putative immunogenic neopeptide's amino acid sequence comprises an amino acid encoded by a nucleotide sequence comprising a distinct amino acid altering nucleotide mutation present in all cells of the malignant neoplasm as determined in claim 4,
C is the probability that the putative immunogenic neopeptide comprises a ligand for MHC in the individual from which the malignant neoplasm is derived, as determined in claim 6,
D is the probability that the neopeptide is immunogenic in the individual from which the malignant neoplasm is derived, as determined in claim 11; and
E is the probability that the neopeptide is resilient toward immune evasion as determined in claim 13,
and determining the composition by excluding from the composition any neopeptide or nucleic acid for which said product does not exceed a predefined threshold value, such as excluding those peptides where said product does not exceed 0.5.

17. The method according to claim 16, wherein the product of at least 2 of A, B, C, D, and E is selected from the group of products of
A and B,
A and C,
A and D,
A and E,
B and C,
B and D,
B and E,
C and D,
C and E,
D and E,
A and B and C,
A and B and D,
A and B and E,
A and C and D,
A and C and E,
A and D and E,
B and C and D,
B and C and E,
B and D and E,
C and D and E,
A and B and C and D,
A and B and C and E,
A and B and D and E,
A and C and D and E,
B and C and D and E, and
A and B and C and D and E.

18. The method according to claim 16, wherein the neopeptides in the composition are those that
have a probability score, which is among the top 50, and/or
have a probability score among the top 50%.

19. The method according to claim 16, which further comprises that only peptides identified by the method according to claim 14 as safe to administer are included in the composition.

20. A method for preparing an immunogenic composition tailored for a patient suffering from a malignant neoplasm, the method comprising sequencing DNA and RNA from malignant cells and at least DNA from normal cells in the patient to identify a set of neopeptides, which comprise neoepitopes, derived from said malignant cells, and subsequently preparing the immunogenic composition by admixing a pharmaceutically acceptable carrier or diluent, and optionally an immunological adjuvant, with
1) at least 1 fusion protein comprising neopeptides from the set but excluding neopeptides from the set, which are not safe to administer when evaluated by the method of claim 15,
2) multiple neopeptides from the set but excluding neopeptides from the set, which are not safe to administer when evaluated by the method of claim 15, or
3) at least one nucleic acid encoding the at least one fusion construct in 1) or the multiple neopeptides in 2).

21. A method for preparing an immunogenic composition tailored for a patient suffering from a malignant neoplasm, the method comprising sequencing DNA and/or RNA from malignant cells and at least DNA from normal cells in the patient to identify a set of neopeptides, which comprise neoepitopes, derived from said malignant cells, and subsequently preparing the immunogenic composition by admixing a pharmaceutically acceptable carrier or diluent, and optionally an immunological adjuvant, with
i) at least 1 fusion protein comprising neopeptides from the set but excluding neopeptides from the set that are not part of the composition determined according to claim 16,
ii) multiple neopeptides from the set that are not part of the composition determined according to claim 16, or
iii) at least one nucleic acid encoding the at least one fusion construct in i) or the multiple neopeptides in ii).

22. (canceled)

23. A method for treating a patient suffering from a malignant neoplastic disease, the method comprising administering an effective amount of an immunogenic composition prepared according to claim 20.

24. A computer or computer system, comprising
a) means for inputting and means for storing nucleic acid sequences,
b) means for inputting and means for storing a qualifier for each nucleic acid sequence input in a, said qualifier indicating whether the inputted nucleic acid sequence originates from malignant cells or non-malignant cells,
c) executable code adapted to generate and store amino acid sequences of expression products encoded by nucleic acid sequences input and stored by the means in a, and which have a qualifier indicating malignant cell origin,
d) executable code adapted to generate and store amino acid sequences of expression products encoded by nucleic acid sequences input and stored by the means in a, and which have a qualifier indicating non-malignant cell origin,
e) executable code adapted to identify amino acid sequences being part of or constituting a sequence generated and stored by the executable code in c, and not being part of or constituting a sequence generated and stored by the executable code in d,
f) executable code for tagging and/or storing each amino acid sequence identified by the executable code in e, including tagging and/or storing information identifying altered amino acid residues relative to the most similar amino acid sequence(s) present in the sequences generated and store by the executable code in d,
g) executable code, which exhaustively compares, for each amino acid sequence tagged or stored by the executable code in f, those amino acid sequences input and stored by the executable code in c, which
all have the same length X, where X is an integer ≥7,
each overlap with the amino acid sequence tagged and/or stored by the executable code in f, and
each include the altered amino acid residue for which information is tagged and/or stored in f, with the amino acid sequences input and stored by the executable code in d,
h) executable code for outputting and/or storing amino acid sequences tagged or stored by the executable code in f while excluding those amino acid sequences for which the executable code in g results in a least one positive comparison.

25. A computer or computer system, comprising
a) means for inputting and means for storing nucleic acid sequences,
b) means for inputting and means for storing a qualifier for each nucleic acid sequence input in a, said qualifier indicating whether the inputted nucleic acid sequence originates from malignant cells or non-malignant cells,
c) executable code adapted to generate and store amino acid sequences of expression products encoded by nucleic acid sequences input and stored by the means in a, and which have a qualifier indicating malignant cell origin,
d) executable code adapted to generate and store amino acid sequences of expression products encoded by nucleic acid sequences input and stored by the means in a, and which have a qualifier indicating non-malignant cell origin,
e) executable code adapted to identify amino acid sequences being part of or constituting a sequence generated and stored by the executable code in c, and not being part of or constituting a sequence generated and stored by the executable code in d,
f) executable code for tagging and/or storing each amino acid sequence identified by the executable code in e, including tagging and/or storing information identifying altered amino acid residues relative to the most similar amino acid sequence(s) present in the sequences generated and store by the executable code in d,
g) executable code, which exhaustively compares, for each amino acid sequence tagged or stored by the executable code in f, those amino acid sequences input and stored by the executable code in c, which
all have the same length X, where X is an integer ≥7,
each overlap with the amino acid sequence tagged and/or stored by the executable code in f, and
each include the altered amino acid residue for which information is tagged and/or stored in f, with the amino acid sequences input and stored by the executable code in d,
h) executable code for outputting and/or storing amino acid sequences tagged or stored by the executable code in f while excluding those amino acid sequences for which the executable code in g results in a least one positive comparison.


SEQUENCE LISTING (10 sequences)

SEQ ID NO: 1 - protein, 9 aa, Artificial sequence
  Model sequence
  misc_feature (4)..(4): Xaa can be any naturally occurring amino acid
ABCXEFGHI

SEQ ID NO: 2 - protein, 15 aa, Artificial sequence
  Model sequence
  misc_feature (8)..(8): Xaa can be any naturally occurring amino acid
KLMNABCXEFGHIST

SEQ ID NO: 3 - protein, 8 aa, Artificial sequence
  Model sequence
  misc_feature (8)..(8): Xaa can be any naturally occurring amino acid
KLMNABCX

SEQ ID NO: 4 - protein, 8 aa, Artificial sequence
  Model sequence
  misc_feature (7)..(7): Xaa can be any naturally occurring amino acid
LMNABCXE

SEQ ID NO: 5 - protein, 8 aa, Artificial sequence
  Model sequence
  misc_feature (6)..(6): Xaa can be any naturally occurring amino acid
MNABCXEF

SEQ ID NO: 6 - protein, 8 aa, Artificial sequence
  Model sequence
  misc_feature (5)..(5): Xaa can be any naturally occurring amino acid
NABCXEFG

SEQ ID NO: 7 - protein, 8 aa, Artificial sequence
  Model sequence
  misc_feature (4)..(4): Xaa can be any naturally occurring amino acid
ABCXEFGH

SEQ ID NO: 8 - protein, 8 aa, Artificial sequence
  Model sequence
  misc_feature (3)..(3): Xaa can be any naturally occurring amino acid
BCXEFGHI

SEQ ID NO: 9 - protein, 8 aa, Artificial sequence
  Model sequence
  misc_feature (2)..(2): Xaa can be any naturally occurring amino acid
CXEFGHIS

SEQ ID NO: 10 - protein, 8 aa, Artificial sequence
  Model sequence
  misc_feature (1)..(1): Xaa can be any naturally occurring amino acid
XEFGHIST